(19) United States
(12) Patent Application Publication
(10) Pub. No.: US 2009/0062202 A1
(43) Pub. Date: Mar. 5, 2009

(54) METHODS OF TREATING AND PREVENTING PRESSURE ULCERS WITH INTRADERMAL AND TRANSDERMAL DELIVERY OF CALCITONIN GENE-RELATED PEPTIDE (CGRP)

(76) Inventor: Richard M. Herman, Scottsdale, AZ (US)

Correspondence Address: THE LUTHER LAW FIRM, PLC, 10575 N 114TH STREET, SUITE 103, SCOTTSDALE, AZ, 85259, US

(21) Appl. No.: 12/198,110

(22) Filed: Aug. 25, 2008

Related U.S. Application Data
(63) Continuation-in-part of application No. 11/745,178, filed on May 7, 2007, now abandoned.
(60) Provisional application No. 60/887,756, filed on Feb. 1, 2007.
(60) Provisional application No. 60/746,849, filed on May 9, 2006.

(51) Int. Cl. A61K 38/17; A61P 17/00

(52) U.S. Cl. 514/12

(57) ABSTRACT

A method of treating ischemic skin pressure ulcers and helping such ulcers heal has the steps of a. debriding the ulcer as necessary to produce a clean, largely uncontaminated surface; and b. applying calcitonin gene-related peptide (CGRP) intradermally or transdermally to the periphery of the ulcer, the CGRP being administered at a dose below a vascular-affecting threshold, thereby enhancing ulcer healing with the anti-ischemic and tissue-healing properties of the CGRP. Also provided is a method of preventing, pressure ulcers of the skin in individuals prone thereto with the steps of a. providing a container with contents comprising CGRP and a vehicle, the CGRP container further comprising connectors for connection to an iontophoresis controller; b. connecting the CGRP container to the iontophoresis controller; c. applying the CGRP container to a body location known to be prone to pressure ulcers; and d. performing an electronic program for forcing CGRP through the skin.

### CROSS REFERENCES TO RELATED APPLICATIONS

This application is a continuation of U.S. application Ser. No. 11/745,178, filed May 7, 2007, which claims the benefit of U.S. Provisional Application No. 60/887,756, filed Feb. 1, 2007 and of U.S. Provisional Application No. 60/887,756, filed May 9, 2006, all of which are incorporated by reference.

### TECHNICAL FIELD

The present invention relates generally to the treatment and prevention of skin pressure ulcers, and is more particularly related to the treatment of a skin pressure ulcers with the neuropeptide calcitonin gene-related peptide (CGRP).

### BACKGROUND

Microvascular dysfunction is a major cause of impaired wound healing and occurs with diabetes, venous stasis, ischemic skin flaps, aging, obesity and spinal cord injury (Kjartannson et al, 1987, 1988; Khalil et al, 1994; Jernberg and Dalsgaard, 1993; Carr et al, 1993; Gherardini et al, 1998a,b; Parkliouse and LeDuesne, 1988; Lundeberg, 1993; Ardron et al, 1991; Appenzeller and McAndrews, 1966). In almost all conditions of microvascular failure, there is functional and anatomical impairment of a group of primary afferent unmyelinated fibers in the skin called “nociceptors, C-fibers” (See Example 1). These C-fibers release neuropeptides, particularly calcitonin gene-related peptide (CGRP), the most abundant peptide in sensory fibers in human skin (Holzer, 1998; Wallengren, 1997) which controls vascular tone of the microvessels and exhibits both trophic and immunomodulatory influences on tissue.

When denervation of C-fibers occur and release of neuropeptides is reduced, wounds can occur which are associated with cellular changes with altered chemotaxis. Moreover, neuropeptide depletion is a common histopathological end-point in various clinical conditions such as Raynaud's Phenomenon, diabetes, obesity, aging, spinal cord injury (SCI) and skin-flaps, all of which exhibit microcirculatory impairment. Under these conditions, CGRP immunoreactivity is markedly decreased (Levy et al, 1989; Schmelz et al, 1997). Depletion of neuropeptides lead to depressed response to skin injury and, hence, reduction in eliciting an inflammatory (vasodilator) reaction as well as diminished trophic and immunomodulatory functions. Anti-ischemic properties have also been emphasized (see below).

Pressure ulcers also cause pain, lost productivity, and huge expenditures for treatment and care (O'Brien et al, 1999). Pressure ulcers are also common in the elderly population, in patients who are bedridden, in patients with SCI and after major orthopedic reconstruction. The prevalence of pressure ulcers (stage 11 and greater) is estimated to be up to 17% in hospitalized patients (Allman, 1989), at least 10% in nursing homes and 20-30% in patients with SCI. The healing rate for stage II ulcers that existed only to the dermis has been as low as 25% at 6 months. The definition of a stage II ulcer is: partial-thickness skin loss involving the epidermis or dermis of both. The ulcer is superficial and presents clinically as an abrasion, blister, or shallow crater. In practical terms, it is not uncommon for patients to be discharged with large unhealed ulcers. With a burgeoning aging population, it is imperative to develop new strategies for treatment and prevention.

Healing of acute wounds (e.g., surgical) occurs sequentially in a timely fashion and without definitive intervention. Usually, platelets enter the wound and secrete growth factors that subsequently recruit macrophages into the wound. Macrophages also release growth factors (Nathan, 1987) that cause endothelial cells to migrate and proliferate in the wound, thereby stimulating angiogenesis and the fibroblasts to synthesize collagen (Brem and Folkman, 1994). The orderly and timely reparative process characteristic of acute wounds results in a sustained restoration of anatomic and functional integrity (Lazarus et al, 1994).

It has been calculated that the total prevalence of venous stasis ulcers, diabetic foot ulcer or pressure ulcers, is between 3 and 6 million (Brem et al, 2000). These physiologically impaired and slow-to-heal wounds place a great burden on the health system, with costs considerably greater than 10 billion dollars/year. These costs do not include the pain and suffering incurred by the patient who may enter the hospital (or nursing home) for a medical disease and who may leave with a sacral ulcer. Among 16 million patients with diabetes in the U.S., an estimated 12% had a history of foot ulcer between the years 2000-2002 (Centers for Disease Control). Approximately 60% of all lower extremity amputations occur among persons with diabetes and, of these, approximately 85% are preceded by a foot ulcer (see also Pecoraro et al, 1990). Limb amputation in patients with diabetes is associated with all increased risk for further amputation, which has a 5-year mortality rate of 39%-68% (Reiber et al, 1995). The direct costs for an amputation range from $20,000-$60,000. When the cost of failed vascular reconstruction and rehabilitation—as well as lost productivity within society—is accounted for, the total costs to society exceed financial analysis. Nevertheless, the costs to society of caring for wounds include the market for wound-care products which is estimated to exceed $7 billion annually (Langley-Hawthorne, 2004). A recent accounting of home health services provided by 7 million patients found that one-third are being seen for the treatment of persistent wounds at a cost of more than $42 billion yearly (Doughty, 2004). Focusing just on one type of chronic wound, the pressure ulcer, total hospital charges were 5.3 times greater than the charges for all other hospitalized patients and the mean length of stay of patients with pressure ulcers was 4.5 times greater; this is in the face of a prevalence of pressure ulcers in the U.S. estimated to be 1.3-3 million (Lyder, 2003) Again, these numbers give only a sense of the magnitude and direct costs of treating wounds, both acute and chronic, but do not account for indirect costs such as lost lives and limbs and the attendant decrease in productivity.

A chronic pressure ulcer wound is characterized by failure to heal in a timely and orderly process, compromising functional and anatomical integrity. Any approach, to be successful, should be relatively simple to administer, with low to minimal side-effects, and should improve wound-healing when compared to non-treated wounds. Healing of chronic wounds chiefly requires concurrent consideration of the inflammatory environment of the wound, persistent infection of the wound, and possible regional ischemia (Isenberg et al, 2005). Addressing such multiple impediments to healing will be necessary to achieve closure of chronic wounds.

Another significant risk factor for pressure ulcers and impaired wound healing is old age. Aged humans reveal major impairment of C-fibers and hence of neurogenic vasodilatation, resulting in failure to appropriately perfuse cutaneous tissue (Helme and McKernan, 1985; Ardron and Helme, 1990). Ardron et al, 1991 showed impairment of microvascular responses in elderly people with varicose leg ulcers. They claim that the nociceptive system of primary unmyelinated nerves probably prevents damage from repetitive minor injuries which go otherwise unnoticed. This nociceptive system is responsible for vasodilatation of the microvessels and has a “trophic” role mediated by the neuropeptides that are released by the C-fibers; the latter functions by acting as growth factors for epidermal cells and fibroblasts. The failure of this same system is found among diabetic patients with leg ulcers (Parkhouse and LeQuesne, 1988; Walmsley et al, 1989). In 1991, Ardron concluded that manipulation of the nociceptive C-fiber system by topical treatment with neuropeptides, may speed-up the healing of chronic skin ulcers.

It would be an advance in the art to provide an effective treatment for the healing of such wounds and for the prevention of the occurrence of such wounds

### SUMMARY

In one embodiment, there is provided a method of treating ischemic skin pressure ulcers and helping such ulcers heal, the method has the steps of a. debriding the ulcer as necessary to produce a clean, largely uncontaminated surface; and b. applying calcitonin gene-related peptide (CGRP) intradermally or transdermally to the periphery of the ulcer, the CGRP being administered at a dose below a vascular-affecting threshold, thereby enhancing ulcer healing with the anti-ischemic and tissue-healing properties of the CGRP. In this method, the skin ulcer is associated with dysfunction or deficiency of small sensory fibers and/or its associated neurotransmitter CGRP. The skin ulcer may be associated with a variety of conditions, including, but not limited to, Raynaud's Phenomenon, secondary Raynaud's Phenomenon, diabetes, spinal cord injury, multiple sclerosis or old age. In the above method, there can be an additional step of monitoring the action of CGRP by determining the local action at the ulcer or by judging the erythema of the surrounding skin and not by plasma levels of CGRP.

Optionally, the method of intradermal application is performed by enhancing penetration of CGRP by iontophoresis. The dose below affecting the vasculature systemically is about 500 picomoles. The method iontophoresis container of CGRP can have a concentration from about 0.001 to about 2.0%, more preferably, about 0.005 to about 1.0%, and even more preferably about 0.008 to about 0.5%. Optionally, the drug mixture contains CGRP at a concentration from about 0.009 to about 0.2% and more preferably at a concentration from about 0.01 to about 0.02%.

In another embodiment, there is provided a method of preventing pressure ulcers of the skin in individuals prone thereto. This method has the steps of a. providing a container with contents comprising CGRP and a vehicle, the CGRP container further comprising connectors for connection to an iontophoresis controller; b. connecting the CGRP container to the iontophoresis controller; c. applying the CGRP container to a body location known to be prone to pressure ulcers; d. performing an electronic program for forcing CGRP through the skin. In this method the concentration of CGRP in the container ranges from about 0.001% to about 2%. Optionally, the body location known to be prone to pressure ulcers is a foot, heel, elbow, or coccyx. Alternatively, the individuals prone to pressure ulcers of the skin include but are not limited to individuals with Raynaud's Phenomenon, pre- and post-diabetes, spinal cord injury, ageing, the elderly, or multiple sclerosis. Other concentrations of CGRP in the container range from about 0.005 to about 1.0%, from about 0.008 to about 0.5%, and from about 0.009 to about 0.2%.

### BRIEF DESCRIPTION OF THE DRAWINGS

A more complete understanding of the implementations may be had by reference to the following detailed description when taken in conjunction with the accompanying drawings wherein:

FIG. 1 is a perspective view of an exemplary perivascular C-Fiber complex.

FIG. 2 shows a graph to demonstrate that during a heat-ramp test, heat-induced flux in the CAP-desensitized subjects (CAP-D) decreased significantly under the thermode (direct flow) and also outside the thermode (indirect flow).

FIG. 3 is a graph to demonstrate that the normal (with vehicle) flare or neurogenic vasodilatory response increases with increasing doses of cathodal current (Flux or AU). Following CAP-desensitization, the dose-response curve is abolished, showing suppression of the nerves mediating the axon reflex.

FIG. 4 is a graph to demonstrate that both direct and indirect flow sites showed a CAP-induced peak flow (flux) that was significantly lower in obese subjects compared to normals.

FIG. 5 is a graph to demonstrate that the latency to CAP-induced direct and indirect (flare) blood flow was significantly prolonged in the obese subjects.

FIG. 6 is a graph to demonstrate that a significant (p<0.001) negative correlation exists between flux (indirect flow) and latency with a correlation coefficient of R₂=0.401 among the obese subjects.

FIG. 7 is a graph to demonstrate that, prior to CAP application (Pre-CAP), the heat pain threshold (HPT) to a step increase in temperature was significantly raised in the obese group. Following CAP (Post-CAP), the HPT was significantly (p<0.001) lowered in both groups, indicating primary hyperalgesia.

FIG. 8 is a graph to demonstrate that, blood flow responses to a ramp increase in temperature from 32° C.-44° C. Note the significant reduction in the flare (neurogenic vasodilatory) response whereas the responses under the thermode were insignificant.

FIG. 9 is a graph to demonstrate that the conduct of the dose-response curve was similar to that described in FIG. 3 in that under normal conditions, blood flow increased gradually with the magnitude of the dose; and under conditions of obesity (like CAP-desensitization) the dose-response curve was markedly diminished.

### DESCRIPTION

It has been surprisingly discovered that chronic pressure ulcers associated with small sensory fiber neuropathy and the consequent failure to produce CGRP can benefit therapeutically from the intradermal administration of CGRP administered around the wound. Further, when administered prior to or during an ischemic state, intradermal administration of CGRP helps prevent pressure ulcers.

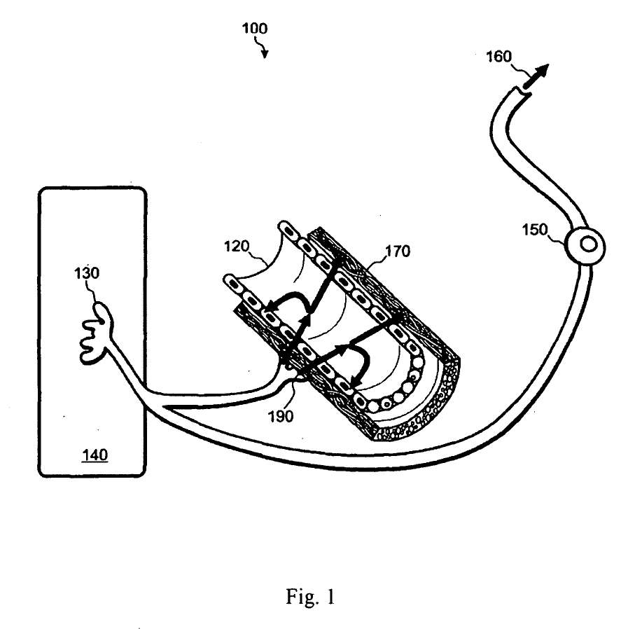

FIG. 1 shows the basis from which was developed the invention. FIG. 1 depicts an example of a perivascular C-Fiber complex 100. Shown in FIG. 1 is a subset of a nerve system in the periphery (e.g., in the skin 140) called C-Fibers 100 in that there are not any myelin or protective coating around it or protective coating. As illustrated by FIG. 1, the endothelium is seen at 120, the polymodal receptor at 130, the skin at 140, the cell body at 150, the direction to the central nervous system (CNS) at 160, the vascular smooth muscle at 170, CGRP at 190, and nitric oxide (NO) at 190. The depicted fibers go to the CNS from left to right as seen at 160 in FIG. 1 to the brain, for instance to convey pain sensation. The fiber also goes to the local blood vessels in the skin and it releases certain chemicals like CGRP. It also affects the endothelium to produce more NO so that there are two vasodilators that work together, ie, CGRP and NO at 190.

Calcitonin Gene-Related Peptide (CGRP) (Wallengren, 1997; Goodman & Iversen, 1986; Wimalawansa, 1996) is a 37-amino-acid peptide, which coexists with Substance P (SP) and Neurokinin A (NKA) in sensory nerve cell bodies, i.e., the dorsal root ganglions (DRGs). CGRP receptors are present on arterial smooth muscle cells and endothelial cells. CGRP is the most abundant neuropeptide in human skin and is co-localized with SP. The N-terminal end of CGRP (amino acids 1-7) has been shown to have CGRP-like activity. This peptide also can be administered with appropriate adjustment of concentration.

The biological effects of CGRP are complex. CGRP potently vasodilates the microcirculation, because CGRP circulates in normal subjects at relatively high concentrations (approximately 25 pmol) and is present in perivascuilar tissues. Struthers et al, 1986 suggested that CGRP controls peripheral vascular tone. Brain et al, 1985 reported that intradermal injections of CGRP in humans at femtoniole doses increased blood flow (flare reaction) that was prolonged and had potency similar to that of prostaglandin-E2. Weidner et al, 2000 also demonstrated that CGRP, administered by microdialysis, produced dose-dependent increases in vasodilatation. The long-lasting and widespread vascular effects of CGRP may reflect gradual diffusion of the peptide which may prolong its actions on both the endothelium and vascular smooth muscles.

Perhaps CGRP plays crucial roles in late inflammation; whereas, SP acts more immediately. Induration may occur with the administration of CGRP and has been ascribed to accumulation of leukocytes (Wallengren and Hakanson, 1987). The CGRP level is stable in the interstitial tissue fluid (Brain and Cambridge, 1996) which could also explain its long-lasting effects. Both Weidner and Wallengren agree that CGRP does not have sensory effects (pain, itch producing), and is not mediated by histamine.

CGRP vasodilates small and large vessels. CGRP produces erythema in femtomol-to-picomol doses which is inhibited by the antagonist CGRP 8-37, indicating a direct effect on the CGRP receptors. CGRP may act by inducing nitric oxide (NO; Khalil and Helme, 1992; Bull et al, 1996). NO may be necessary for either the release of CGRP from sensory nerves or its subsequent pathway. CGRP enhances endothelial cell proliferation and may therefore participate in angiogenesis during physiological and pathophysiological events such as ischemia, wound healing and inflammation. CGRP also modulates immune cell functions. For example, it enhances phagocytosis by cultured peritoneal mouse macrophages. Finally, the availability of neuropeptides to trigger cellular responses is effectively controlled by neuropeptide-specific peptidases.

CGRP has been shown to help the healing of ischemic flaps and experimental wounds. Electrical stimulation with and without direct application of CGRP also aids in wound healing. In normal rats, flaps fail to heal when sensory nerves are desensitized by pre-treatment with CAP, suggesting the role of neuropeptides in delayed wound healing.

One of the most important mechanisms regarding the action of CGRP is its anti-inflammatory property (Raud et al, 1991). CGRP inhibited edema-promoting action of inflammatory mediators, e.g., histamine, leukotrine, and in vivo. CGRP was effective in the nanomolar dose range and was mimicked by activation of sensory nerves (C-fibers) by acute capsaicin (CAP) which caused endogenous CGRP-like immunoreactivity (IR). This anti-inflammatory response is surprising because it contrasts with the pro-inflammatory reaction of CGRP released from sensory nerves. In this regard, CGRP participates in “neurogenic vasodilatation”, also an essential mechanism in wound repair (see Richards et al, 1997; Khalil and Helme, 1996). Thus, both pro-inflammatory and anti-inflammatory reactions to exogenous CGRP (intradermal) and endogenous CGRP (released by acute CAP; Holzer, 1998) appeared to act in wound healing.

CGRP, administered intradermally to wounds in rats, had a trophic effect on healing by increasing the contraction rate (Engin, 1998). Engin ascribed improved healing of wounds by CGRP as a form of local action on sensory terminals near the defect to produce its trophic action rather than to the hyperesponsiveness of exogenous CGRP on sensitive ischemic tissue. Moreover, CGRP can have a direct action on endothelial cells, thus releasing NO, and on vascular smooth muscle, both increasing its trophic and immunomodulatory functions. CGRP is effective in the presence of C-fiber inaction as observed after pre-treatment with CAP, and under conditions of aging, diabetes and other conditions with small fiber neuropathy (see Example 1).

Not wishing to be bound by any particular theory, it is proposed that pre-terminal changes in nociceptors (C-fibers) also occur and are most likely due to degeneration of epidermal fibers which are predominantly CAP-sensitive C-fibers and may not be due to altered axonal transport. Functional impairment of C-fibers and reduced epidermal nerve density have been noted among subjects with Raynaud's Phenomenon, diabetes, obesity, aging and spinal cord injury, all of whom are at high risk for pressure ulcers and delayed healing of wounds.

As observed in animals and humans with venous stasis and diabetic ulcers, TENS treatment with low frequency (e.g., 8 Hz) and high intensity current accelerated wound healing in aged rats and rats pre-treated by CAP by peripheral activation of sensory nerves (Khalil and Mehri, 1996). Khalil demonstrated that direct infusion of CGRP in blister wounds caused wound healing. However, her experiments did not reveal whether CGRP would be effective in the treatment of pressure ulcers which are characterized by ischemia which is the major cause of difficulty of treating most pressure ulcers.

The therapeutic efficacy of replenishing CGRP is limited by methods of administration (Brain and Grant, 2004). In addition to the methods of administration described above, there are other injection-free methodologies utilizing passive (e.g., a polymer which can be used on the surface of the skin) or active (e.g., iontophoresis) transdermal administration of CGRP for the treatment/prevention of neuropathic ulcers. Given the relatively large molecular weight of ˜3800 Daltons, the penetration of CGRP is achieved by systems that disrupt the barrier mechanisms of the epidermis so that CGRP can reach the injured tissue and the abundant sub-epidermal plexus of C-fibers and microvascular vessels.

Passive transdermal systems are employed to diffuse the drug through the skin where the drug can act locally or penetrate the capillaries for systemic effect. This technology is only effective with small molecular drugs which can be utilized with skin patches containing such substances as nicotine, clonidine, and nitric oxide (NO).

In active transdermal systems, drug molecules of significant molecular weight are forced through the skin. By using an applied force (e.g., ultrasound or electrical stimulation-iontophoresis already described), active transdermal systems are capable of delivering proteins and other large molecules through the skin and, if necessary, into the bloodstream. Transdermal delivery of CGRP alone has never been tested by iontophoresis (or by any other means) to treat pressure ulcers. In addition, no one has suggested or tested administering CGRP to the pressure ulcer periphery.

Administration by iontophoresis is particularly attractive for the treatment of wounds because it is noninvasive and thus does not contribute to the infectious load of the hospitalized patient. Iontophoresis usually employs two containers, a positively charged container (anode) which repels a positively charged drug into the body, and a negatively charged container (cathode) which repels a negatively charged drug into the body. CGRP, being a positively charged ion, will be driven by a positively charged current. Both containers are in operative contact (e.g., wired) to a controller permitting the operator to select a program for drug delivery. In general, the longer the period, the lower the charge required. However, the treatment period needs to adapt to the patient's intensive and complex therapeutic regimen. Active anodal treatment may be pulsed on/off over a prescribed period of time.

Systemic administration may have undesirable side-effects including heart rate and blood pressure changes. The advantage of local administration using either intradermal or transdermal methods is that lower concentrations of CGRP will produce good clinical results without systemic side effects.

To summarize the above issues, pressure ulcers of the skin are a common outcome of various human conditions (see below). In most instances, they are related to a small sensory fiber neuropathy (SSFN). Small sensory nerves (often referred to as C-fibers or nociceptors) primarily convey pain signals to the central nervous system and act by an axon-reflex to control microvascular vasodilatation by releasing calcitonin gene-related peptide (CGRP) from their perivascular terminals. CGRP is the most abundant neuropeptide in these neurons; hence, SSFN is accompanied by depletion of CGRP. Small sensory nerve fibers and CGRP are reduced in human conditions such as (1) pre- and post-diabetes; (2) Raynaud's Phenomenon; (3) spinal cord injury and multiple sclerosis; (4) ageing and elderly; and (5) venous ulcers.

It is critical to recognize that pressure ulcers in these conditions are associated with impaired microcirculation and tissue ischemia at vulnerable anatomical sites (e.g., weight bearing sites). Among all the sensory neuropeptides, these events can be modified solely by the administration of CGRP. While vasodilatation and trophic actions of a number of sensory neuropeptides (e.g., substance P or SP) have been ascribed to the healing of wounds, these effects may not occur in skin pressure ulcers associated with ischemia ((Knight et al, 1990). In fact, although there has been much interest in the therapeutic use of SP for wounds (e.g., blister wounds) in animal experiments, it is clear that SP's vasodilatory action is transient in nature and there is no evidence that SP has an anti-ischemic action. Tachyphylaxis also interferes with SP's therapeutic usefulness (Franco-Cereceda et al, 1987). Thus, the wound healing properties of CGRP, i.e., anti-ischemia, vasodilatation, anti-inflammation, should not be confused with those of other sensory neuropeptides nor with other vasodilators in general.

The administration of CGRP has been principally conducted by invasive means (intravenous and intradermal, i.d.). Khalil however demonstrated that CGRP delivered directly to an induced blister-base wound in elderly or capsaicin-treated rodents caused enhanced healing. While this is an important observation since the animals sustained a SSFN, it nevertheless did not demonstrate whether CGRP would be effective in the treatment of pressure ulcers. Khalil's wound model does not reveal an ischemic state which is a profound pathophysiological event in wounds related to SSFN, e.g., pressure ulcers in diabetes, spinal cord injury.

The mechanism(s) by which CGRP increases survival of ischemic tissue may differ from its vasodilatory effect (Heden et al, 1989; Kjartansson and Dalsgaard, 1987). Within the context of wound healing of non-ischemic or ischemic tissue, the direct application of CGRP to non-ischemic tissue would be understandable given the vasodilatory and anti-inflammatory properties of the molecule. However, in conditions such as pressure ulcers where ischemia is the early pathological process prior to skin ulcers or can surround the skin ulcers, it is most likely that CGRP will have to be delivered around the wound, where blood vessels still exist.

The critical issue is the transdermal (non-invasive) administration of a large molecule weighing ˜3800 Daltons. Iontophoresis is the process of “driving” ions into the skin by application of electric current. In recent years, iontophoresis has been used clincially to deliver low molecular weight substances such as lidocaine and dexamethasone through the skin. Rossi et al (2007) demonstrated that the skin can be penetrated by CGRP by a technique such as iontophoresis. However, CGRP's vasodilatory action was demonstrated only in normal humans. The dose-response, anti-ischemic and wound healing characteristics were not revealed.

Iontophoresis has also been used to treat ischemic ulcers due to Raynaud's Phenomenon (with histamine) and diabetic ulcers (with zinc, prostaglandin E) (Abramson et al, 1967; Comwall, 1981; Sakurai and Yamamura, 2003) with varying results. Gherardini et al, 1998 showed that CGRP+Vasoactive Intestinal Polypeptide caused healing of venous stasis ulcers in humans. Transdermal delivery of CGRP alone has never been tested by iontophoresis (or by any other means) to treat pressure ulcers, particularly when administered around the wound. While we are not claiming that iontophoresis is the only transdermal method of administering CGRP, it is an attractive concept given that the electric current alone, placed about diabetic ulcers, may have an additional healing potential, in part by increasing release of CGRP in intact small sensory neurons (Lundeberg, 1993). However, in the presence of SSFN associated with depleted release of CGRP, the addition of CGRP for iontophoresis should augment considerably the effect of the current, thus serving to “replace” the neuropeptide and to act upon CGRP-sensitive ischemic tissue. In the presence of skin pressure ulcers, intradermal or transdermal, delivery of CGRP is anticipated to occur after debridement and signs of infection are under clinical control.

Intradermal injections are another method of delivering CGRP. This technique would be useful in interpreting the “proof of principle” that CGRP can prevent ischemic attacks and reduce tissue loss as in Raynaud's Phenomenon. There are multiple novel methods of applying intradermal injections via “painless” needle injection techniques.

Although we regard the treatment potential for specific wounds, namely pressure ulcers, to be important, prevention of skin ulcers in human subjects with SSFN associated with ischemia, impaired circulation, cellular inflammation and depressed immunomodulation (e.g., diabetes, Raynaud's Phenomenon, elderly) is also a principal focus of this application. This concept is based upon the observations that after surgery of island flaps in rats, intra-arterial CGRP enhanced survival of the flaps when administered prior to ischemia, as well as during ischemia and after ischemia (Westin and Heden, 1988). Moreover, Jansen et al, 1999 reported that intraperitoneal CGRP, at doses below those influencing skin flap blood flow, caused tissue survival of the flaps due to its anti-inflammatory action against induced neutrophil recruitment when delivered prior to an ischemic event.

Thus, we propose that CGRP can be successful in reducing the prevalence of skin pressure ulcers and, hence, reduce morbidity, health care costs, and social/vocational consequences of pressure ulcers. CGRP can be administered transdermally or intradermally to skin areas at high risk for pressure ulcers with ischemia (or during ischemia)(e.g., heels and metatarsals of diabetics, fingers of subjects with Raynaud's Phenomenon; coccyx of subjects with spinal cord lesions). Admittedly, efforts towards prevention by constantly altering the position of body parts (by manual means or devices such as special mattresses) have helped reduce the incidence of pressure ulcers. Yet pressure ulcer prevention remains an enormous task for the patient and the health delivery system.

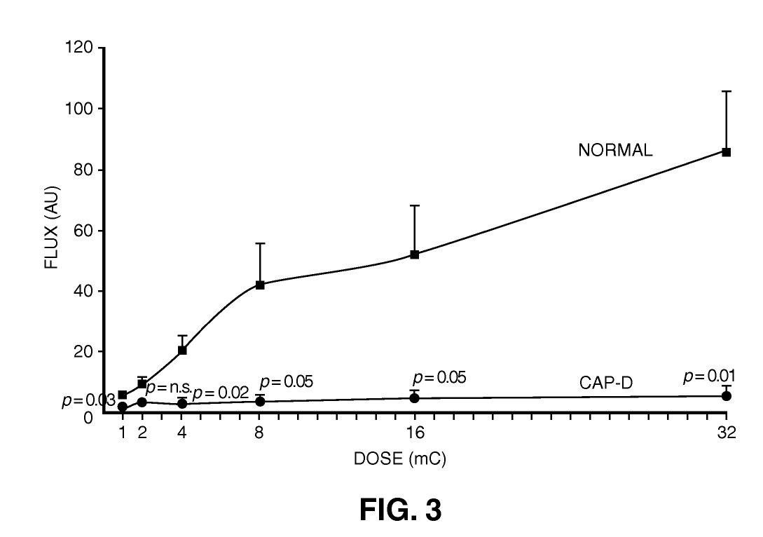

By way of example, and not by way of limitation, relative to a human hand, a treatment contemplated for Raynaud's disease and phenomenon is illustrated in FIG. 3.

### EXAMPLE 1

### Small Fiber Neuropathy in Obesity

Somatic cutaneous small sensory fiber neuropathy (SSFN) is an early manifestation of Impaired Glucose Tolerance and Diabetes Mellitus and/or insulin resistance among obese subjects and is often associated with pain, wound occurrence, and impaired wound healing. However, it is unclear as to whether SSFN is prevalent among obese individuals who have delayed wound healing, but no glucose and/or insulin dysregulation.

We set out to study whether there is hypofunctioning of stimulated capsaicin (CAP)-sensitive nerves (small sensory fibers-SSF) in obese subjects with/without hyperglycemia and hyperinsulinemia.

Fifty-eight morbidly obese and 15 lean subjects were recruited for small fiber testing of the forearm in a cross-sectional study. Hyperglycemia was observed in 35 obese subjects. Of 25 obese subjects, hyperinsulinemia was noted in 15, 14 of whom were hyperglycemic. No subjects demonstrated symptoms/signs of neuropathy over the hairy skin of the forearm. In fact, a neurological examination revealed that 37 subjects were asymptomatic in the legs and only 4 complained of a neuropathic pain in the foot. Virtually all subjects were exposed to a set of CAP-sensitive tests and measures with CAP desensitization procedures. These tests, conducted with supine patients in bed, examined the two principle roles of cutaneous SSFs, namely conveying pain signals to the CNS and controlling local neurogenic vasodilatation (flare; axon-reflex).

Heat-induced pain was assessed by verbal reports of sensation after accommodation and heat, and CAP. Transcutaneous stimulation-induced blood flow was measured by laser Doppler flowmetry with probes placed at the site of stimulation and 1 cm remote from the site, the latter to evaluate flare latency and intensity of flare.

Specific noxious and non-noxious stimuli are applied to the skin to activate nociceptive afferent neurons to induce a spreading hyperemia, which is visible as a neurogenic vasodilatory, or flare, response. Assessment of the axon-reflex flare by noxious heat- and CAP-stimulation and by non-noxious transcutaneous electrical stimulation can be used to measure C-fiber function. While objective measurement of the flare reaction can be accomplished by visual assessment, introduction of laser Doppler flowmetry (LDF) has made it possible to monitor the flare response continuously in a small area of the skin, thereby facilitating the study of the axon-reflex in terms of flux.

Continual exposure to topical CAP leads to desensitization of nociceptive afferent neurons and, thus, increases the heat pain threshold and virtually abolishes the neurogenic vasodilatory response to noxious mechanical, thermal or irritant chemical stimulation as well as to non-noxious stimulation. Such outcomes provide evidence that these tests can be used to assess the function of C-fibers and SSFNs. CAP-induced desensitization is ascribed to morphological damage of Epidermal Nerve Fibers (ENFs) which contain a high concentration of nociceptive afferent neurons and CGRP.

### Methodology

Subjects: Fifteen (15) normal and fifty-eight (58) morbidly obese subjects were recruited. The morbidly obese subjects had a BMI>35 and a waist/hip ratio consistent with visceral obesity. Table 1 lists the population characteristics.

TABLE 1
Population Characteristics

| | Normal | | Obese | |
|---|---|---|---|---|
| Characteristics | Subjects n = 15 | | Subjects n = 58 | |
| | Mean | Range | Mean | Range |
| Age (years) | 32.40 | 20.00-57.00 | 45.30 | 29.00-68.00 |
| Males BMI (kg/m²) | 22.80 | 20.00-25.00 | 48.50 | 36.00-68.00 |
| Females BMI | 21.30 | 19.00-25.00 | 48.60 | 35.00-65.00 |
| (kg/m²) | | | | |
| Males Waist/Hip | 0.89 | 0.83-0.92 | 1.00 | 0.89-1.10 |
| Ratio | | | | |
| Females Waist/Hip | 0.76 | 0.72-0.79 | 0.83 | 0.76-1.10 |
| Ratio | | | | |
| Glucose (mg/dl) | 85.00 | 81.00-88.00 | 137.00 | 68.00-339.00 |
| HbAlC | | | 6.90 | 4.80-11.10 |
| Cholesterol | 174.00 | 101.00-256.00 | 197.00 | 130.00-270.00 |
| Triglyceride | 74.00 | 43.00-120.00 | 229.00 | 45.00-505.00 |
| Hyperglycemia | Number | Percent | Number | Percent |
| Diabetic | 0 | 0% | 21 | 36% |
| IGT | 0 | 0% | 14 | 24% |
| Hyperlipidemia | 6 | 40% | 38 | 66% |
| Sleep Apnea | 0 | 0% | 15 | 26% |
| Tobacco Use | 0 | 0% | 14 | 24% |

Significant depression of pain and flare responses were observed in the obese subjects in all but one test. Decreased pain and flare responses were noted in all subjects without hyperglycemia and hyperinsulinemia. Age negatively correlated with CAP-induced flare in both the obese and normal groups.

Normal subjects were excluded if they exhibited hyperglycemia, hyperinsulinemia, sleep apnea or tobacco use; whereas, obese subjects were accepted with various conditions related to the metabolic syndrome. Peripheral vascular disease or an ankle-brachial index of <0.9 excluded a subject. All qualified subjects received a clinical neurological examination of the peripheral nervous system of the upper and lower extremities.

Tests were conducted in a 24-26° C. room with the subject reclined in a hospital bed with one or two pillows under the head. Right and left forearms were tested alternately with the stimuli being placed away from any prior hyperemic skin. The hairy skin of the proximo-lateral forearm was cleansed with an alcohol swab. Prior to each test, 60 seconds of baseline temperature and blood flow were recorded. These values were subtracted from peak blood flow values to yield a “net” flux.

Cutaneous blood flow was measured by laser Doppler flowmetry (LDF), using a DRT 4. Laser Doppler Flowmeter system (Moor Instruments of Devon, England). A fiber optic probe directed laser-generated light of a wavelength of 780 nm-820 nm to the skin surface. As the light reflected from moving blood cells, it underwent a shift in frequency (Doppler Effect) dependent on the number and velocity of the moving blood cells. Blood flow was measured in arbitrary units (AU). The probe was placed into a recess in the center of a stimulating chamber (e.g., PERSPEX, thermode) to measure “direct flow” and in a recess 10 mm outside the edge of the stimulator to measure “indirect flow” or the axon-reflex. This probe configuration was used for all tests. Direct and indirect flow was measured under three modes of stimulation: heat via a contact thermode, topical CAP application via a passive iontophoretic chamber, and transcutaneous electrical stimulation via an active iontophoretic chamber.

Before testing for heat stimulation, each subject underwent a training trial to ensure their prompt verbal report of the various sensations accompanying rising temperature (e.g., warm, itchy, hot, burning). Heat was delivered by a circular brass contact thermode (10 mm diameter) centered in a 35 mm stabilizing collar placed on the forearm. A probe was placed in the center of the thermode and a second probe was placed 1 cm from the thermode. Two forms of stimulation were used: heat-ramp and heat-step.

In the heat-ramp procedure, the thernode temperature was fixed at 32° C. for 1 minute followed by a 25-second ramp to 44° C. that was then maintained for 20 minutes. The subject reported continuously on perceived sensations. The time of each report and the maximum net hyperemic response as determined by LDF were recorded.

In the heat-step procedure, basal blood flow was monitored at resting skin temperature and later at 32° C. The temperature was raised in increments of 2° C. per minute until a maximum temperature of 48° C. was reached. Sensory responses (specifically, heat pain threshold, HPT) and peak net flows at each increment were documented.

A second heat-step test followed an acute application of topical CAP to the skin to measure sensitization by HPT, i.e., primary hyperalgesia. Two normal and obese subjects complained that the pungency from the CAP was extreme and, as a consequence, further testing with the heat-step test for hyperalgesia was not conducted.

Topical CAP: CAP, 8-methyl-N-vanillyl-6-nonenamide, is a vanilloid neurotoxin that targets somatic small primary afferent neurons. CAP (1% in 75% ethanol, 25% saline) was applied topically for 45 minutes through a central chamber (300 .mu.l capacity, 1 cm diameter) in a clear circular plastic PERSPEX container adhered to the skin. Throughout the application, perceptual responses and peak flow values were obtained. The vehicle was applied at different skin sites to six normal subjects.

Transcutaneous Electrical Stimulation: A non-noxious, constant cathodal current was delivered by a platinum ring electrode in a central iontophoretic chamber. The central reservoir of the iontophoretic chamber was filled with methylcellulose (2%), an inert vehicle for iontophoretic drug delivery. An indifferent electrode was applied to the wrist. Progressive increases in current dose (millicoulombs, mC) were delivered using a current of 0.2 mA applied for 10, 20, 40, 80 and 160 sec. These represented doses of 2, 4, 8, 16, and 32 mC. A post-stimulus interval of 180 seconds followed each of the first four doses; a 240 second interval followed the highest dose. Peak flow at the end of each stimulus period was recorded.

CAP-induced Desensitization: Eight (8) normal subjects were recruited to ascertain the degree of CAP sensitivity of each of the tests described above. Each subject applied topical CAP to one forearm and thigh, and the vehicle to the opposing limb in 45 minute applications, 3-4 times daily for 5 days.

Statistical Analysis: Group mean data for continuous variables, such as blood flow, temperature and latency were compared using unpaired t-tests (HPT, flare, latency) and one-way analysis of variance with Tukey's post-hoc multiple comparison tests for differences in dose response curves (electrical stimulation). Data for individual participants were standardized for each dependent variable using Z-scores, and Pearson's R2 correlation values with 95% confidence interval were used to detect relationships between variables. All statistical analyses were performed by means of SPSS statistical software (version 11.5.0). Statistical significance was determined by a two-tailed assessment. All p-values were designated within the figure (n.s.=p>0.05).

Results: Demographics

Table 1 identifies the pertinent variables associated with age, sex, BMI, Waist/Hip Ratio, and levels of glycemia and lipidemia in the two population samples. Insulin values were obtained from 10 normals (all normal values) and 25 obese subjects. Of the 25 obese subjects, 15 were hyperinsulinemic, 14 of which were also hyperglycemic (IGT=9; DM=6). Hyperglycemia was noted in 35 obese subjects and in none of the normal subjects. Of these 35 obese subjects, 14 had IGT and 21 had DM. Of the normal subjects 40% had hyperlipidemia.

The neurological examination of 55 obese subjects revealed the following: 25 had no symptoms or signs of neuropathy; 11 reported no symptoms but demonstrated small fiber impairment; 1 reported no symptoms but showed large and small fiber impairment; two groups of 7 reported non-painful symptoms, usually in the foot, with either involvement of small fibers only or both large and small fibers; 4 reported painful feet associated with small and large fiber neuropathy. There were no symptoms or signs of neuropathy over the area of skin tested. Thirty obese subjects had small sensory fiber neuropathy on clinical examination.

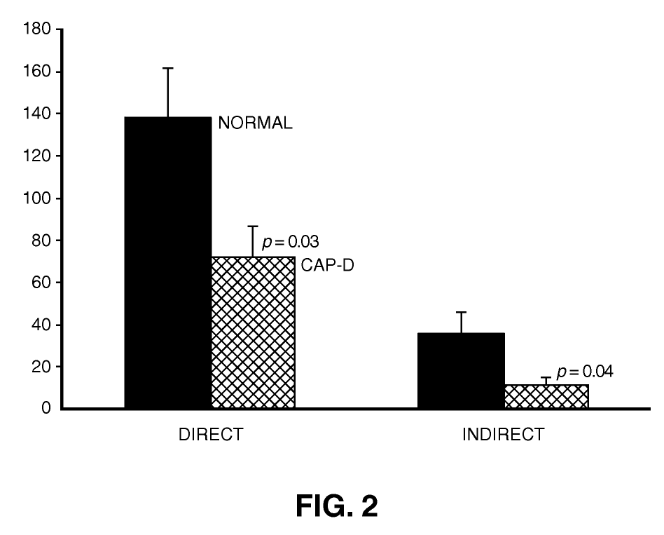

The method of CAP-induced desensitization calls for topical CAP, or control vehicle, to be applied to normal subjects (n=8) 3-4 times daily for 5 days. When compared to vehicle alone, this regimen of CAP-induced desensitization (CAP-D) to heat, acute CAP and transcutaneous electrical stimulation was pronounced for each stimulus based upon the following observations: 1. HPT increased to greater than the maximum temperature (48° C.). 2. During the heat-ramp test, heat-induced flux decreased significantly under the thermode (direct flow) and also outside the thermode (indirect flow) (FIG. 2). 3. An acute CAP challenge produced no pungency and virtually no increased blood flow (not illustrated). 4. At the indirect site, a non-noxious transcutaneous electrical (cathodal) stimulation utilizing the vehicle produced a rising dose (mC)-response (flux) curve. Following CAP-D the curve was flattened indicating an abolished neurogenic flare (FIG. 3). 5. Consequently, heat, CAP and electrical stimulation-induced responses of pain (pungency) and/or flare were considered CAP-sensitive neurogenic responses indicative of small fiber impairment.

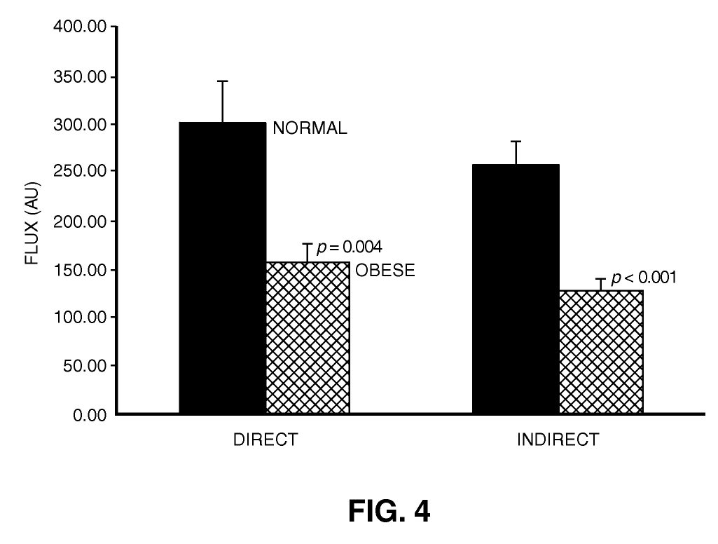

To determine the acute Effects of CAP, quality of sensation and blood flow were determined. Using criteria for CAP-induced pungency (heat etc.), normal subjects reported a 90% pungency level with approximately 80% perceiving burn/pain and 20% reporting a stinging/prickly sensation. Obese subjects reported only a 45% pungency level described as burn/pain in all but one subject. Six (6) subjects did not perceive any change in sensation (e.g., itch, warm). In normal subjects, and obese, pungency and flare were not elicited by the vehicle alone (FIG. 4). Both direct and indirect flow sites showed a CAP-induced peak flow that was significantly lower in obese subjects compared to normals. The mean peak direct and indirect flow values for the normal and obese subjects are depicted in Table 2.

TABLE 2
Summary Data

| | Direct | | Direct | | Indirect | | Indirect | |
|---|---|---|---|---|---|---|---|---|
| Test | Normal | | Obese | | Normal | | Obese | |
| CAP | Mean | SEM | Mean | SEM | Mean | SEM | Mean | SEM |
| Flux (AU) | 299 | 44.00 | 159 | 17 | 257 | 27 | 128 | 13 |
| Latency (min) | 11 | 2 | 21 | 2 | 12 | 1 | 26 | 2 |
| Heat Ramp | | | | | | | | |
| Flux (AU) | 313 | 50 | 226 | 19 | 155 | 32 | 59 | 8 |
| Heat Step HPT | | | | | | | | |
| Pre-CAP | 44.8° C. | 0.6 | 46.7° C. | 0.2 | | | | |
| Post-CAP | 37.1° C. | 1.2 | 39.8° C. | 0.9 | | | | |
| Hyperalgesia | | | | | | | | |

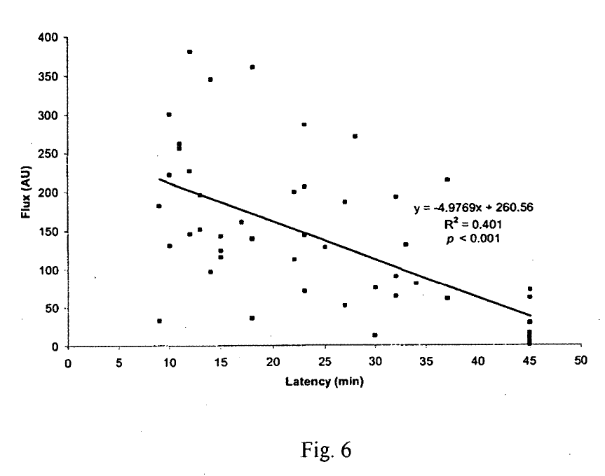

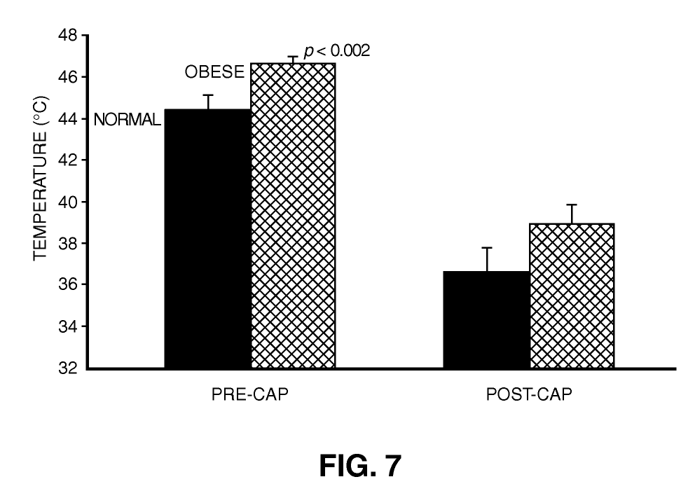

As for latency, both direct and indirect flow sites showed a CAP-induced vasodilatory response latency that was significantly delayed in obese subjects compared to normals. The mean latency for direct and indirect flows is summarized in Table 2. Thirty one (31) obese subjects demonstrated significantly prolonged latencies (e.g., >20 min) to the flare response (greater than 2 standard deviations from the normal mean). Among these 31 subjects, 20 did not report pungency. Furthermore, 18 subjects revealed very low flow amplitudes, defined as a flux <100. Eleven (11) subjects demonstrated a flare latency at or near the maximum exposure time of 45 min or not at all. Of these 11 subjects, 8 reported no pungency. The normal subjects exposed to the vehicle had no delayed response. FIG. 6 depicts a significant (p<0.001) negative correlation between flux (indirect flow) and latency to the flare with a correlation coefficient of R²=0.401. Regarding CAP-induced Hyperalgesia to Heat, CAP caused a marked primary hyperalgesic response to heat in both the normal and obese populations (FIG. 7, Post-CAP). Both groups demonstrated a significant fall (p<0.001) in the HPT with an average of 7° C. (see Table 2). Approximately 80% of the obese population (n=43/53) and 90% of the normal population (n=11/13; 2 complained of excessive pain before the heat probe could be applied) demonstrated a pronounced hyperalgesic response of 5° C.-16° C. Of 9 obese subjects who demonstrated a IIPT at the peak temperature or did not report pain, all but one revealed a long latency/low amplitude blood flow response to acute CAP application.

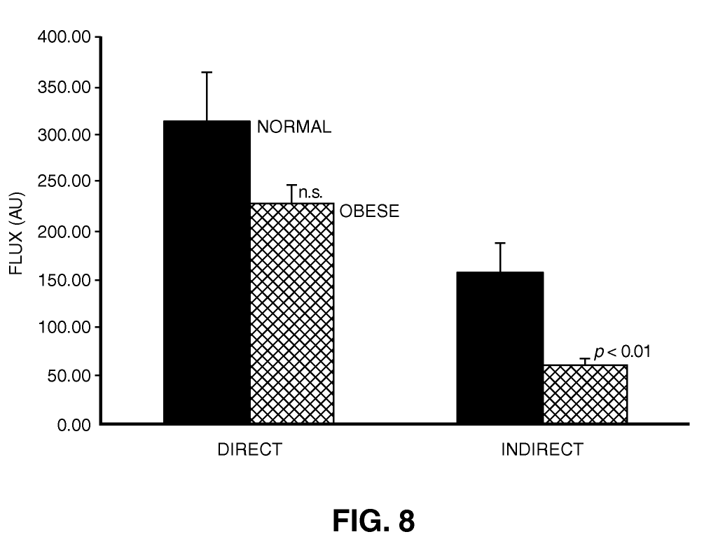

Responses to Heat-Step and Heat-Ramp Increases 1. Heat-Step Increases. Step increases in heat every 2° C. from 32° C. to 48° C. were associated with a significantly (p=0.007) increased HPT in the obese group (Table 2 and FIG. 7 Pre-Cap). 2. Heat-Ramp Increases. A heat-ramp stimulus from 32° C. to 44° C. (at an average speed of 2° C./sec) caused a significant reduction in flare intensity of >50% (FIG. 4); whereas, there was no significant difference between the two groups when comparing blood flow responses at the direct site (Table 2; FIG. 8). While the flare reactions were profoundly impaired, perceptual reports from obese subjects indicated that about 45% voiced “pain/burn” sensation to or at 44° C., a value similar to that of the normal subjects. Approximately 15% (n=8) of the obese subjects had no change in sensation.

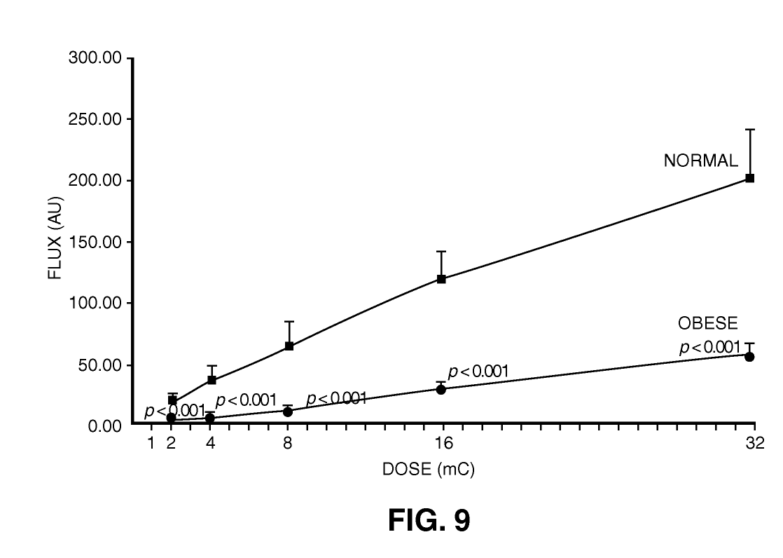

Dose-Response Relationships During Transcutaneous Electrical Stimulation: Dose (mC)-response (flux) curves were obtained during incremental increases in duration of stimulation at the same current intensity (0.2 mA). In both groups, direct and indirect flux increased as a function of current dose. Increasing charge or dose (mC) was associated with significantly lower flux at each dose in the obese group at both direct (not illustrated) and indirect sites (FIG. 9).

Correlations with BMI, Age, and Hyperglycemia: Pearson's correlation coefficients were used to examine the relationships between reported pain (in terms of threshold or presence of pungency) and flare with BMI, age, and hyperglycemia in each of the two groups. The only significant observation was that of age and CAP flare response (two-tailed, R²=−0.114; p=0.017) for the obese group. Age and CAP-induced flare for the normal group also indicated a negative correlation but the coefficient did not yield significant figures, most likely due to the small population sample. Using frequency histograms of Z scores for each or the sum of 6 sets of pain/flare data (i.e., HPT prior to and following CAP application, CAP pungency and flare latency and magnitude, and flare magnitude with electrical stimulation), the potential role of hyperglycemia and hyperinsulinemia was analyzed. Pearson correlations did not reveal significance between fasting hyperglycemia and normoglycemia (<100 mg/dl of glucose; <5.5 HbAlC) nor between hyperinsulinemia and normoinsulinemia.

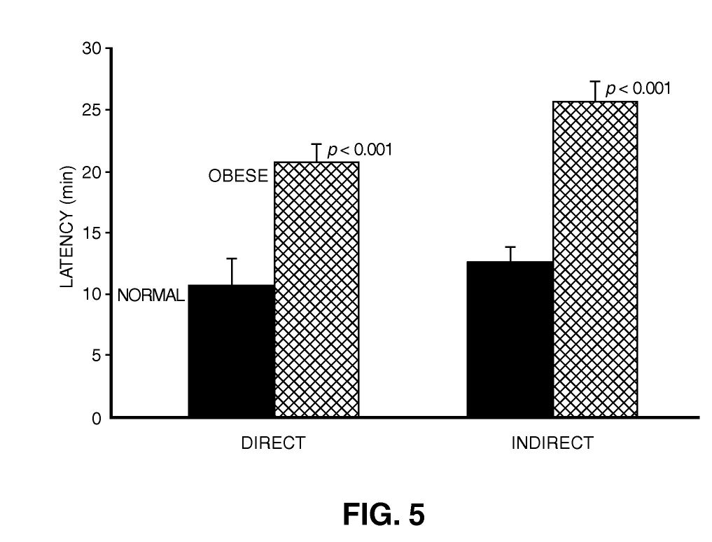

In conclusion, the application of CAP-sensitive tests to a large group of morbidly obese subjects reveals that small fiber, notably C- and A.delta.-nociceptor, neuropathy (SSFN) is common in this population. The following observations are indicative of functional impairment of cutaneous nociceptor activation: 1. Decreased pungency, magnitude and latency of flare to acute CAP treatment (FIGS. 4-6). 2. Raised HPT to heat-step and decreased flare to heat-ramp stimuli (FIGS. 7, 8). 3. Depressed flare (FIG. 9) to transcutaneous electrical stimulation.

Additionally, a number of issues are identified: Utilization of CAP-sensitive tests suggests that SSFN is a generalized disorder; in contrast, our clinical examination using heat/cold stimuli reveals a length-dependent or dying-back process, which may imply two distinct pathological processes. The presence of hyperalgesia following CAP treatment in subjects with obvious SSFN suggests that the thermode size was sufficient to activate some nociceptors, including presumably “silent” nociceptors (e.g., mechano-insensitive nociceptors), which would lead to summation of impulses by central neurons to cause pain (neurogenic hyperalgesia). If depressed blood flow measured under the thermode (“direct flow”) during a heat-ramp test is an indicator of microangiopathy, obese subjects do not reveal a microangiopathic process (at least in the upper extremity) to account for the microcirculatory changes observed with CAP treatment and electrical stimulation. Hyperglycemia (IGT, diabetes) is a potential contributing factor in SSFN among the obese subjects but only about 70% were considered hyperglycemic (a 2-hour post-prandial test might prove more useful). A potential causative factor is oxidative stress which is raised in obesity, diabetes, insulin resistance, and neuropathy. Importantly a major proportion of the obese patients had impaired c-fiber function, which is a proven precursor to pressure ulcers. The obese too could benefit from therapy to prevent the development of pressure ulcers.

### EXAMPLE 2

### Prevention

Individuals with scleroderma often have secondary Raynaud's Phenomenon (SRP), with increased sympathetic outflow to the digits, and thickening of the skin. There is an extreme response to cold due to sympathetic outflow, which results in skin ulcers, leading to amputations. Pharmacotherapy for SRP has not been satisfactory. Currently calcium channel blockers and prostaglandins are used. Iloprost may be the first choice for individuals with critical digital ischemia and/or digital ulceration, but this drug must be administered for extended periods of time daily for at least five days in a hospital setting. Troublesome side effects due to systemic vasodilatory action principally occur during the infusion. For individuals with frequent attacks, calcium channel blockers have been the gold standard, even though they have a low efficacy rate. Individuals with SRP continue to require procedures for skin ulceration, infection, gangrene and amputation—at great cost. Not wishing to be bound by a particular rationale, I propose that the problem in SRP is that the sympathetic outflow overwhelms the amount of CGRP present. Ordinarily CGRP would counteract the problematic ischemia but is not sufficient in SRP. Therefore, we will administer CGRP to the digits of individuals with SRP to prove this hypothesis.

To volunteers with SRP, intradermal (id) administration of CGRP to a single digit is performed. Patients are tested for a substantial and persistent increase in skin blood flow within 60 min after the injection. They also will be tested for prevention of an ischemic attack caused by cold provocation of die fingers. CGRP concentrations range from about 2×10⁻⁶ μg/μl to about 4×10⁻³ μg/μl. When the CGRP is provided in 50 ul syringes, these doses range from about 0.25 to about 50 pmoles. The 50-pmolar dose is one tenth of a dose known to cause no systemic effects such as lower blood pressure.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within the meaning and range of equivalency of the claims are to be embraced within their scope.

1. A method of treating ischemic skin pressure ulcers and helping such ulcers heal, the method comprising
a. debriding the ulcer as necessary to produce a clean, largely uncontaminated surface;
b. applying calcitonin gene-related peptide (CGRP) intradermally or transdermally to the periphery of the ulcer, the CGRP being administered at a dose below a vascular-affecting threshold, thereby enhancing ulcer healing with the anti-ischemic and tissue-healing properties of the CGRP.

2. The method of claim 1 wherein the skin ulceration is associated with dysfunction or deficiency of small sensory fibers and/or its associated neurotransmitter CGRP.

3. The method of claim 3 wherein the skin ulceration is associated with Raynaud's Phenomenon, secondary Raynaud's Phenomenon, diabetes, spinal cord injury, multiple sclerosis or old age.

4. The method of claim 1 further comprising the step of
c. monitoring the action of CGRP by determining the local action at the ulcer or by judging the erythema of the surrounding skin and not by plasma levels of CGRP.

5. The method of claim 1 wherein step b of intradermal application is performed by enhancing penetration of CGRP by iontophoresis.

6. The method of claim 1 wherein the dose of CGRP is below 500 picomoles.

7. The method of claim 5 wherein the administration method of iontophoresis has the step of providing a container of CGRP at a concentration from about 0.001 to about 2.0%.

8. The method of claim 7 wherein the drug mixture contains CGRP at a concentration from about 0.005 to about 1.0%.

9. The method of claim 7 wherein the drug mixture contains CGRP at a concentration from about 0.008 to about 0.5%.

10. The method of claim 7 wherein the drug mixture contains CGRP at a concentration from about 0.009 to about 0.2%.

11. The method of claim 7 wherein the drug mixture contains CGRP at a concentration from about 0.01 to about 0.02%.

12. A method of preventing pressure ulcers of the skin in individuals prone thereto, the method comprising the steps of
a. providing a container with contents comprising CGRP and a vehicle, the CGRP container further comprising connectors for connection to an iontophoresis controller;
b. connecting the CGRP container to the iontophoresis controller;
c. applying the CGRP container to a body location known to be prone to pressure ulcers; and
d. performing an electronic program for forcing CGRP through the skin.

13. The method of claim 12, wherein the concentration of CGRP in the container ranges from about 0.001% to about 2%.

14. The method of claim 12, wherein the body location known to be prone to pressure ulcers comprises a foot, heel, elbow, or coccyx.

15. The method of claim 12, in which the individuals prone to pressure ulcers of the skin comprise individuals with Raynaud's Phenomenon, pre- and post-diabetes, spinal cord injury, ageing, the elderly, obesity or multiple sclerosis.

16. The method of claim 12 wherein the concentration of CGRP in the container ranges from about 0.005 to about 1.0%.

17. The method of claim 12 wherein the concentration of CGRP in the container ranges from about 0.008 to about 0.5%.

18. The method of claim 12 wherein the concentration of CGRP in the container ranges from about 0.009 to about 0.2%.
